(19) United States
(12) Patent Application Publication
(10) Pub. No.: US 2004/0161438 A1
(43) Pub. Date: Aug. 19, 2004

(54) Cosmetic preparations comprising active ingredients in microcapsules

(75) Inventors: Nadja Jungmann, Essen (DE); Peter Lersch, Dinslaken (DE); Ute Schick, Koln (DE); Yvonne Schiemann, Essen (DE); Christian Weitemeyer, Essen (DE)

Correspondence Address: SCULLY SCOTT MURPHY & PRESSER, PC, 400 GARDEN CITY PLAZA, GARDEN CITY, NY, 11530

(73) Assignee: GOLDSCHMIDT AG, ESSEN (DE)

(21) Appl. No.: 10/774,935

(22) Filed: Feb. 9, 2004

(30) Foreign Application Priority Data
Feb. 18, 2003 (DE) 103 06 604.7

(51) Int. Cl. A61K 7/00

(52) U.S. Cl. 424/401

(57) ABSTRACT

Cosmetic preparations including one or more active ingredients in a microencapsulation whose encapsulation material is permeable and/or degraded in the pH range of the skin, wherein the core material is free from porous materials are provided.

### FIELD OF THE INVENTION

The present invention relates to cosmetic preparations comprising one or more active ingredients in a microencapsulation whose encapsulation material is permeable and/or is degraded in the pH range of skin, wherein the core material is free from porous materials.

### BACKGROUND OF THE INVENTION

When it is a question of achieving and promising particular effects of cosmetic products, the ingredients are a central theme. The high standard of supplied ingredients and raw materials in cosmetic formulations is being continuously broadened since consumers are interested in high-quality and effective products which can counteract the effects of aging. In this respect, the interest of the cosmetics manufacturer is also directed to active ingredients which are able to revitalize the skin or to offer protection against the consequences of light aging. If such substances have in the past primarily served for the smoothing and moisturization of the skin, then the substances are nowadays supplemented by a large number of different materials with a physiological effect. Examples thereof are vitamins, fruit acids and also ceramides. In this connection, the nature and method of stabilizing such active ingredients is also of increasing importance. In cosmetics there is a great interest in active ingredients, which can be stably stored in aqueous as well as water-containing systems.

For the purpose of using one or more cosmetic skin active ingredients and/or aroma substances and/or food supplements, it is desirable to encapsulate these or to provide them with a coating. In particular, this measure is suitable for thermolabile, oxidation-sensitive substances and also readily volatile fragrances.

Encapsulations are of use when active ingredients are to be protected and made to last for longer if they are to penetrate well into the skin, be uniformly distributed and released in a controlled manner.

The aim of a microencapsulation can therefore serve different purposes, such as that of controlling the release behavior of an active ingredient, the coating of liquid substances, a masking or protection of the core material, the reduction in the volatility, and the improvement in compatibility with other substances, e.g., for compounding.

According to the present invention, the term “microcapsules” is understood as meaning particles and aggregates which comprise an internal space or core that is filled with a solid, gelled, liquid or gaseous medium and are surrounded (encapsulated) by a continuous coating of film-forming polymers. These particles are preferably small in size.

In addition, the microscopically small capsules can comprise, in distributed form, one or more cores in the continuous encapsulation material, consisting of one or more layers.

Preference is given to single-core microcapsules with a continuous shell.

The production of microcapsules has been described in detail in the literature of the prior art and is accessible by means of known reactive and nonreactive processes, such as solvent vaporization, precipitation processes, coazervation, interfacial polycondensation, and etc.

Solvent vaporization is used for producing reservoir and matrix systems and includes, inter alia, spray-drying and drum-coating.

In the precipitation process, the polymeric wall material is dissolved in a water-miscible solvent and the active ingredient to be encapsulated is dispersed therein. The dispersion is then introduced into the continuous aqueous phase with intensive thorough mixing.

Coazervation is understood as meaning the separation of a colloidal dispersion (liquid/liquid or solid/liquid) in a phase with a high content of liquid dispersed material (coazervate) and a phase with a low content brought about by external influences.

In contrast to the other microencapsulation processes used, such as solvent vaporization or coazervation, which use already prepared polymers as coating materials, in the interfacial polycondensation technique the shell is formed from the corresponding monomers only during the course of the encapsulation process.

Encapsulation materials are usually natural, semisynthetic or synthetic inorganic and, in particular, organic materials.

Natural organic materials are, for example, gumarabic, agar, agarose, maltodextrins, alginic acid or its salts, e.g., sodium or calcium alginate, liposomes, fats and fatty acids, cetyl alcohol, collagen, chitosan, lecithins, gelatin, albumin, shellac, polysaccharides, such as starch or dextrin, cyclodextrins, sucrose and waxes.

Semisynthetic encapsulation materials are, inter alia, chemically modified celluloses, in particular, cellulose esters and ethers, e.g., cellulose acetate, ethyl cellulose, hydroxypropylcellulose, hydroxy-propyl-methyl-cellulose and carboxymethylcellulose, and also starch derivatives, in particular, starch ethers and esters.

Synthetic encapsulation materials are, for example, polymers, such as amino resins, polyacrylates, polyamides, polyvinyl alcohol, polyvinylpyrrolidone, or organopolysiloxanes.

The modification relates, for example, to the degree of crosslinking of the polymers which essentially determines the permeability of the shell, but also the chemical composition of the polymer which is responsible for the compatibility between encapsulation material and core material.

The microcapsules can vary with regard to shape and size within wide limits depending on the preparation process, although the microcapsules are preferably approximately bulb- or sphere-shaped and, depending on the substances present inside them, have a diameter in the nanometer range (cannot be detected visually, “invisible”) up to the millimeter range.

“Invisible” microcapsules preferably have a diameter in the range from 20 to 500 nm, preferably 50 to 200 nm.

The visible capsules are larger than 500 micrometers in diameter and colored due to encapsulated pigments. They are found in shower gels, hair care products and dental creams.

The microcapsules used according to the present invention are preferably in the range from 1 to 1000 μm, in particular from 10 to 200 μm. Some of the processes for the preparation of microcapsules are notable for the fact that severe preparation conditions with reaction temperatures above 100° C. are required. Such processes are not suitable for the encapsulation of cosmetic active ingredients since the active ingredient to be encapsulated is often largely, or in unfavorable cases even completely, decomposed under such conditions.

The release of the substances from the microcapsules usually takes place while the preparations comprising them are being used, as a result of disintegration of the shell caused by mechanical, thermal, chemical or enzymatic action. The aforementioned opening variants also have an effect on the valuable biological activity of the encapsulated ingredients.

In cosmetic formulations for the treatment of normal skin, but in particular sensitive, irritated skin and very particularly in baby care, it is, however, often problematical or impossible to use such microencapsulated active ingredients for obvious reasons.

Of particular importance for applications in cosmetic formulations is the degree of penetration of the microencapsulated active ingredients into the skin—associated with a depot effect in the horny layer or the epidermis. Deep penetration (transdermal permeation) is reserved here instead for pharmaceutical applications.

In skincare, it must also be ensured that the acid protective mantel of the skin is not damaged by unsuitable additives, but is retained and assisted, i.e., the “natural” ambient conditions are largely retained.

The surface of the skin is covered with a thin film of sebum, sweat and amino acids. The significance of this acidic property of the surface of the skin is expressed in the so-called acid protective mantle. The term “acid protective mantle” means that the protective film of sebum and water on the skin's surface itself acts like a very weak acid (pH value).

More recent research results demonstrate that the acidic pH of the horny layer plays an essential role for the formation and structuring of the epidermal lipids and thus the permeability barrier. These investigations show that an acidic medium is important for:

activating the enzymes for the synthesis of important epidermal lipids,

forming the double layers of the lipid membrane,normalizing the horny layer barrier following mechanical or chemical damage.

A closer inspection of the constituents of the hydrolipid film reveals why this protective film was referred to in 1928 for the first time by Schade and Marchionini as an acid protective mantle:

sweat comprises lactic acid and various amino acids,

sebum comprises free fatty acids,

amino acids and pyrrolidonecarboxylic acid arise from the keratinization process.

The uppermost layer of the skin is made up of cells layered one above the other which lie on top of one another loosely, comparable with roof tiles. The material for this layer is actually skin waste from dead and flat horny cells. These are stuck together by skin fats or lipids and moisture. Because fat repels water, this fat/moisture mixture in the outer skin acts outwardly like a raincoat. At the same time it prevents our skin from vaporizing too much moisture from inside by body heat. Water-soluble harmful substances have virtually no chance of penetrating this barrier. The same is true, however, for water-soluble care substances. Fat-soluble active ingredients are more readily able to penetrate into the skin.

Requirements which are ideally placed on an encapsulation system for cosmetic active ingredients are therefore manifold. As well as a gentle and rapid inclusion process, which should be easy to carry out and suitable for the preparation of microcapsules with constant quality, the active ingredient to be encapsulated should be coated as completely as possible because only then is adequate protection ensured. Preferably, the microcapsules are prepared in a simple one-step process and the wall material used is commercially available polymers which are distinguished by a defined chemical composition. When choosing the polymer material, it should be taken into consideration that no undesired skin reactions are caused and that the type of release mechanism can be adjusted so that the acid protective mantel of the skin is not impaired.

### SUMMARY OF THE INVENTION

An object of the present invention is to provide cosmetic preparations for the treatment of skin which comprise the active ingredients in a microencapsulation. Such preparations should also satisfy a wide variety of the requirement criteria already mentioned and release the active ingredient continuously following application to the skin without impairing the acid protective mantel of the skin.

Particular advantages are afforded here by a polymer system in which cosmetic active ingredients have only low solubility since in such a polymer mixture the active ingredient has a great endeavor to leave the polymer. The low density of the system additionally provides for short diffusion pathways.

Japanese patent application, JP-A-06-105069, discloses a process for the preparation of pH-sensitive microcapsules. In this connection, it is stated that the pH-sensitive polymer is firstly dissolved and is then dispersed with the material to be encapsulated and a porous carrier material, such as, for example, silica. The suspension obtained in this way is added to a dispersion medium and, with vaporization of the solvent or as a result of phase separation in the dispersion medium, microcapsules with diameters between 0.001 and 1000 micro-meters are formed. It is pointed out that the inadequate stability of the microcapsules is improved through the addition of a porous carrier material. Since the active ingredient is taken up into the pores of the carrier material, no internal decomposition of the microcapsules and no impairment of the cosmetic formulation by the active ingredient can arise. The types of polymer which are mentioned as being useful are, within the scope of a general listing, both alkali- and also acid-labile polymers.

Surprisingly, it has been found that through the use of Eudragit® E100, a copolymer based on 2-dimethylaminoethyl methacrylate, methyl methacrylate and n-butyl methacrylate, as an encapsulation material for the active ingredients, it is possible to prepare microcapsules for incorporation into cosmetic formulations which can be prepared without the use of additional agents and carrier materials and without the use of mechanical energy for making the capsule wall material permeable.

The non-use of porous carrier materials has the great advantage that, when applied to the skin, no residues of hard core materials (ghosts) remain which can impair the physiological compatibility or bring about cosmetically undesired effects, such as an unpleasant feel on the skin.

As a result of the use of the porous carrier material, larger capsules have to be used in order to be able to absorb an amount of active ingredient adequate for a physiological effect. This is not the case for the microcapsules described here, which are free from carrier materials. In contrast, the microcapsules without filler can provide bioactive components which, firstly, comprise the active ingredient fractions, based on the starting material, in amounts which exceed the amounts given in JP-A-07096166 and, secondly, have a reduced size, as a result of which easier and pleasant cosmetic application on the skin is achieved.

It has also been found that, by mixing this base polymer with any other polymers, the pH-controlled release behavior is retained if the proportion of base polymer constitutes more than 20% by weight. As a result of the mixing with other polymers, preferably with polymers functionalized with ionizable groups, properties such as biodegradability, the release behavior of the active ingredients and also the preparation costs can be influenced in a favorable manner.

The present invention therefore provides cosmetic preparations comprising one or more active ingredients in a microencapsulation whose encapsulation material is permeable and/or is degraded in the pH range of skin, wherein the core material is free from porous materials.

### DETAILED DESCRIPTION OF THE INVENTION

In one preferred embodiment of the present invention, the compositions comprise microcapsules in amounts from 0.1 to 10% by weight, in particular from 0.2 to 8% by weight, and more particularly from 0.5 to 5% by weight.

The encapsulation materials used according to the present invention are copolymers based on 60 to 40% by weight of 2-dimethylaminoethyl methacrylate, 20 to 30% by weight of methyl methacrylate and 20 to 30% by weight of n-butyl methacrylate and copolymers based on in each case 50% by weight of methyl methacrylate and ethyl acrylate. These compounds and their preparation are described in DE-B-1 617 751 and EP-A-0 181 515.

The corresponding commercial products are available under the trade name EUDRAGIT® from Röhm GmbH, Darmstadt.

By varying the degree of copolymerization, the composition of the polymer can be adjusted such that the resulting encapsulation material is soluble, swellable and permeable above a pH of 5.

The copolymers based on 50% by weight of 2-dimethylaminoethyl methacrylate, 25% by weight of methyl methacrylate and 25% by weight of n-butyl methacrylate which are preferably used according to the present invention are notable for the fact that they have average molar masses of from 50,000 to 250,000 g/mol, where the materials preferably used should have average molar masses in the range from 100,000 to 200,000 g/mol, in particular 130,000 to 170,000 g/mol.

It is also possible to use this base copolymer in a mixture with other natural or synthetic polymers provided it is ensured that the pH-controlled opening of the resulting mixtures is retained.

Typical examples of active ingredients as are used in the field of cosmetic preparations are surfactants, cosmetic oils, perlescent waxes, stabilizers, antimicrobial active ingredients, anti-inflammatory active ingredients, plant, yeast and algae extracts, vitamins, vitamin derivatives and complexes, amino acids and amino acid derivatives, bioactive lipids, such as cholesterol, ceramides and pseudoceramides, deodorants, anti-perspirants, antidandruff agents, UV light protection factors, antioxidants, preservatives, insect repellants, self-tanning agents, tyrosinase inhibitors (depigmentation agents), perfume oils and dyes. Preferred active ingredients are those which, in nonencapsulated form, either can not be stably worked into formulations or at least do not remain stable over prolonged storage periods.

The cosmetic preparations for the treatment of the skin are formulations customary in practice which comprise the constituents typical for the particular intended use in the customary amounts. These formulations are known to one skilled in the art and can thus be used provided the pH is outside the range in which disintegration of the encapsulation material occurs.

The examples below are intended to illustrate the subject-matter of the invention in more detail:

Polymer:

Copolymer based on 50% by weight of 2-dimethylaminoethyl methacrylate, 25% by weight of methyl methacrylate and 25% by weight of n-butyl methacrylate and an average molar weight of about 150,000 g/mol (EUDRAGIT® E 100, Röhm GmbH):

### EXAMPLE 1

5 g of polymer were dissolved in 30 ml of acetone. 0.1 g of aluminum tristerate (i.e., an emulsifier) and 0.5 g of tocopherol (an active ingredient) were then added. This solution was stirred for 20 minutes at 10° C. and 250 rpm and then the solution was added to 200 ml of 10° C. cold paraffin oil. The resulting reaction solution was stirred for a further 4 hours at 190 rpm or 500 rpm then filtered off and washed with 50 ml of n-hexane. The spheres obtained were dried at room temperature.

Result:

Uniformly shaped spheres which had an average diameter of 600 μm were produced. The spheres did not stick together and thus they were present individually. pH-controlled opening of the capsules was possible using hydrochloric acid (pH 5.5), and also in a buffer solution which had been adjusted to a pH of 5.0. By adding the buffer to the spheres, the active ingredient was seen to emerge after about 15 minutes under a microscope. After a further 45 minutes, the spheres dissolved slowly and the active ingredient became clearly visible.

### EXAMPLE 2

1 g of polymer was dissolved in 30 ml of acetone. 0.1 g of aluminum tristerate, as an emulsifier, and 0.5 g of lipoic acid, as active ingredient, were then added. This solution was stirred for 20 minutes at 10° C. and 250 rpm and then added to 200 ml of 10° C. cold paraffin oil. The resulting reaction solution was stirred for a further 4 hours at 200 rpm, then filtered off and washed with 50 ml of n-hexane. The spheres obtained were dried at room temperature.

Result:

Uniformly shaped spheres which had an average diameter of 200 μm were produced. The spheres did not stick together and thus they were present individually. pH-controlled opening of the capsules was possible with hydrochloric acid (pH 5.5), and with buffer (pH 5.0). By adding the buffer to the spheres, the active ingredient was seen to emerge after about 10 minutes under a microscope. After a further 30 minutes, the spheres dissolved slowly and the active ingredient became clearly visible.

### EXAMPLE 3

5 g of polymer were dissolved in 30 ml of acetone. 0.5 g of emulsifier (e.g., aluminum tristearate) and 0.5 g of methanol were then added. This solution was stirred for 20 minutes at 10° C. and 250 rpm and then it was added to 200 ml of 10° C. cold paraffin oil. The resulting reaction solution was stirred for a further 4 hours at 250 rpm, then filtered off and washed with 50 ml of n-hexane. The spheres obtained were dried at room temperature.

Result:

Uniformly shaped spheres which had an average diameter of 150 μm were obtained. The spheres did not stick together and thus they were present individually. pH-controlled opening of the capsules was possible using hydrochloric acid (pH 5.5), and using buffer (pH 5.0). By adding the buffer to the spheres, of the active ingredient was seen to emerge after 10 minutes under a microscope. After a further 30 minutes, the spheres slowly dissolved and the active ingredient became clearly visible.

### EXAMPLE 4

2.5 g of polymer were dissolved with 2.5 g of poly(dl-lactide-co-glycolid) in 30 ml of acetone. 0.1 g of emulsifier (e.g., aluminum tristearate) and 0.5 g of vitamin E were then added. This solution was stirred for 20 minutes at 10° C. and 250 rpm and then it was added to 200 ml of 10° C. cold paraffin oil. The resulting reaction solution was stirred for a further 4 hours at 250 rpm and then filtered off and washed with 50 ml of n-hexane. The spheres produced were dried at room temperature.

Result:

Uniformly shaped spheres which had an average diameter of 300 μm were produced. The spheres did not stick together and thus they were present individually. pH-controlled opening of the capsules was possible using hydrochloric acid (pH 5.5), and using buffer (pH 5.0). By adding the buffer to the spheres, the active ingredient was seen to emerge after about 12 minutes under a microscope. After a further 40 minutes, the spheres dissolved slowly and the active ingredient became clearly visible.

pH-Controlled Release of the Active Ingredient Using Buffer Solution (in vitro):

The resulting spheres from experiment 1 were added to buffer solution pH 5.0 (Merck) and measured photometrically (wavelength 332 nm) after various times. The absorbance was directly proportional to the percentage of active ingredient released.

Initial Weight:

0.5 g of spheres/50 ml of buffer solution

| Time | Release % |
|---|---|
| 20 sec | 53.5 |
| 1 min | 69.0 |
| 2 min | 85.9 |
| 2.30 min | 89.4 |
| 3 min | 91.7 |
| 3.30 min | 96.4 |
| 4 min | 100 |

pH-controlled release of the active ingredient on the skin (in vivo):

A classic skincare cream based on a W/O emulsion was prepared. For this purpose, an oil phase comprising 45.6 g of paraffin oil and 2.4 g of ABIL EM 90 (Goldschmidt) was initially introduced and stirred with a MIG stirrer at 450 rpm. To this was added a water phase comprising 147.2 g of water, 4.0 g of glycerol and 0.8 g of NaCl over the course of 3 min and then the mixture was homogenized for 3 min at 1300 rpm. Finally, citric acid was used to adjust the pH to 6.5, and 1% by weight of the microcapsules according to the present invention containing lipoic acid as active ingredient (prepared as described in example 2) were stirred into the finished cream. This formulation was stored at room temperature and at elevated temperature at 40° C. over a period of a total of 2 months. During this time, samples were taken weekly, the shape of the capsules was analyzed microscopically and, following filtration of the capsules, the cream formulation was monitored with regard to its content of released lipoic acid by means of HPLC analysis. The result was that, under the described conditions, the microcapsules remained stable over the entire storage period and likewise no escape of active ingredient into the cream was observed. pH-induced opening of the spheres was checked by applying the cream to the skin of a total of 6 test persons and then covering the areas with adhesive tape.

After a contact time of one hour, the adhesive tape was removed from the skin with the capsules or capsule residues adhering thereto and viewed under a microscope. Under the microscope it was possible to clearly see that an opening of the polymer capsules under the acidic pH conditions of the skin had taken place.

An identical result could also be achieved by removing relatively large capsules directly from the cream and, likewise after having been fixed to the skin with adhesive tape, by removing said capsules from the skin after one hour and then analyzing them microscopically. Here too, the hoped-for effect was found since no intact capsule material could still be found.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A cosmetic preparation comprising one or more active ingredients in a microencapsulation, said microencapsulation having an encapsulation material that is permeable into skin, is degraded in a pH range of skin, or both and a core material that is free from porous materials.

2. The cosmetic preparation of claim 1, wherein the preparation further comprises non-microencapsulated active ingredients.

3. The cosmetic preparation of claim 1, which comprises 0.1 to 10% by weight, based on the total formulation, of microcapsules.

4. The cosmetic preparation of claim 1, which comprises microcapsules whose encapsulation material comprise copolymers of 2-dimethylaminoethyl methacrylate, methyl methacrylate and n-butyl methacrylate.

5. The cosmetic preparation of claim 1, which comprises microcapsules whose encapsulation material comprises 60 to 40% by weight of 2-dimethylaminoethyl methacrylate, 20 to 30% by weight of methyl methacrylate and 20 to 30% by weight of n-butyl methacrylate.

6. The cosmetic preparation of claim 1, which comprises microcapsules whose encapsulation material comprises copolymers of 2-dimethylaminoethyl methacrylate, methyl methacrylate and n-butyl methacrylate with an average molar mass of from 50,000 to 250,000 g/mol.

7. The cosmetic preparation of claim 1, further comprising additional fractions of encapsulation materials selected from the group of gumarabic, agar, agarose, maltodextrins, alginic acid, alginates, fats, fatty acids, cetyl alcohol, collagen, chitosan, lecithin, gelatin, albumin, shellac, polysaccharides, celluloses, cellulose esters, cellulose ethers, starch ethers, starch esters, polyacrylates, polyamides, polyvinyl alcohols and polyvinylpyrrolidone.

8. The cosmetic preparation of claim 1, further comprising substantially spherical microcapsules whose diameter is from 1 to 1 000 μm.

9. The cosmetic preparation of claim 8, wherein the substantially spherical microcapsules are bulb-shaped or ball shaped.

10. The cosmetic preparation of claim 1, further comprising microcapsules whose encapsulation material decomposes at a pH value of between about 4.6 and about 6.0.
